(19) United States
(12) Patent Application Publication
(10) Pub. No.: US 2021/0338687 A1
(43) Pub. Date: Nov. 4, 2021

(54) MIFEPRISTONE ORAL FORM FOR ITS USE IN CERVIX RIPENING AND LABOUR INDUCTION

(71) Applicant: DISPHAR INTERNATIONAL BV, Hengelo (NL)

(72) Inventors: Sophie Rolande VAN TOMME, Baarn (NL); Helene Marie Virginie HERMAN, Paris (FR); Hans SCHRAM, Marolles En Brie (FR)

(73) Assignee: DISPHAR INTERNATIONAL BV, Hengelo (NL)

(21) Appl. No.: 17/085,399

(22) Filed: Oct. 30, 2020

(30) Foreign Application Priority Data
May 4, 2020 (AT) 28071
May 4, 2020 (CZ) D20043531
May 4, 2020 (DE) 20 2020 102 472.8
May 4, 2020 (RO) 2020 00012

(51) Int. Cl. A61K 31/567; A61K 9/00; A61K 45/06; A61P 15/04

(52) U.S. Cl. CPC A61K 31/567; A61P 15/04; A61K 45/06; A61K 9/0053

(57) ABSTRACT

The invention relates to an oral dosage form of mifepristone of mifepristone oral formulation characterised in that said formulation comprises an amount selected from the group consisting of 75 mg, 150 mg or 300 mg mifepristone, and said formulation is for its use in the induction of labour and cervix ripening in a woman being at least 37 weeks pregnant.

### CROSS-REFERENCE TO RELATED APPLICATIONS

This patent application claims the benefit of Austrian Patent Application No. 28071, filed on May 4, 2020, Czech Patent Application No. D20043531, filed on May 4, 2020, German Patent Application No. 20 2020 102 472.8, filed on May 4, 2020, and Romanian Patent Application No. 2020 00012, filed on May 4, 2020, the disclosures of which are incorporated herein by reference in their entireties for all purposes.

### FIELD OF THE INVENTION

The present invention relates to novel dosages of a mifepristone oral form for its use in cervix ripening and labour induction.

### TECHNICAL BACKGROUND

The average length of human gestation is 280 days, or 40 weeks, from the first day of the woman's last menstrual period. A pregnancy is considered early term 37 weeks up to 39 weeks of gestation, full term 39 weeks up to 41 weeks of gestation, and late term 41 weeks up to 42 weeks of gestation.

Induction of labour is defined as the process of artificially stimulating the uterus to start labour. Induction of labour can be needed in various situations and notably when there is a risk for the baby's health or the mother's health, when the waters has broken but the cervix is unfavourable, or when the mother is more than 40 weeks pregnant and the labour does not start naturally.

The first step can be a manual dilation of cervix in order to manually rupture the amniotic membranes and induce uterus contractions depending on the gestational age and the emergency of the delivery.

Another common technique for labour induction is to insert a balloon catheter into the cervix to help it dilate so that the baby can pass through the birth canal. A balloon catheter is a long, rubber tube with an inflatable balloon on one end that can be filled with air or sterile water. When the balloon inflates inside the cervix, it puts pressure on the cervical cells, helping it dilate and increasing the tissue's response to oxytocin and prostaglandins, and hence increase the uterus contractions.

The induction of labour can also be performed by administering prostaglandins (PGE2 and PGF2a) or prostaglandin analogues. Prostaglandins are hormones, produced throughout the body, which induce labour. They are applied locally to the vagina as tablets, gels, suppositories and pessaries to reduce side-effects. The dose, number of doses, and time between doses vary considerably. Sustained release pessaries reduce the need for repeat doses and so the number of vaginal examinations. Dinoprostone, a PGE2 prostaglandin, ripens the cervix and induce uterine contractions. Misoprostol is a PGE1 prostaglandin analogue and is commonly used to start labour. Prostaglandins and prostaglandin analogues cause uterine contractions and the ripening, effacement or thinning, of the cervix.

Oxytocin is a peptide hormone naturally produced by hypothalamus and released by the posterior pituitary gland. It causes cervical dilation and uterine contractions. However, oxytocin is not effective when the cervix is unfavourable.

If the cervical status is unfavourable, a ripening process is generally employed prior to induction to shorten the duration of induction and maximize the possibility of vaginal delivery. It is part of the induction of labour.

A cervix is considered unfavourable when it is not adequately prepared for a vaginal delivery of a new-born child. Within the present application, it is considered that the cervix is unfavourable when the Bishop's score is of less than 6 or 6. A favourable cervix is one with a Bishop's score more than 6.

Otherwise stated, the induction of labour comprises the cervical ripening or cervical maturation in order to facilitate the induction of labour, which consists in inducing uterine contractions. Indeed, cervical ripening, also called cervical effacement or cervical maturation, consist in softening the cervix. Prior to effacement, the cervix is like a long bottleneck, usually about four centimetres in length. Throughout pregnancy, the cervix is tightly closed and protected by a plug of mucus. When the cervix effaces, the mucus plug is loosened and passes out of the vagina. As effacement takes place, the cervix then shortens, or effaces, pulling up into the uterus and becoming part of the lower uterine wall. Effacement may be measured in percentages, from zero percent (not effaced at all) to 100 percent, which indicates a paper-thin cervix. Effacement is accompanied by cervical dilation. Cervical dilation is the opening of the cervix.

Mifepristone is recognized as a component of safe abortion and is included to the WHO Model Lists of Essential Medicines (WHO Model Lists of Essential Medicines 21^{st} List (2019) (available at https://www.who.int/medicines/publications/essentialmedicines/en/). The administration of 200 mg of mifepristone orally has been studied previously. In a study, this dosage has been proven to improve the Bishop score after 24 hours vs. placebo and to reduce the use of prostaglandin, but these findings were not statistically significant (D. Wng et al, Mifepristone for Preinduction Cervical Ripening Beyond 41 Weeks' Gestation: A Randomized Controlled Trial, Obstetrics & Gynecology, 96 (4) October 2000, pp. 843-548). A clinical trial found that mifepristone was efficient on inducing cervical ripening and labour in full-term pregnancy. According to this trial, as mifepristone has shown the ability to induce cervical ripening in term pregnancy, comparative studies are needed to find out in which situations, gestational ages, indications and combinations with other methods of labour induction its usage might be most effective and safe. (O. R. Baev et al., Outcomes of mifepristone usage for cervical ripening and induction of labour in full-term pregnancy. Randomized controlled trial, European Journal of Obstetrics & Gynecology and Reproductive Biology 217 (2017) 144-149). However, a study comparing the efficiency of mifepristone at the dosage 50, 100, 200, 400 and 600 mg of mifepristone vs placebo concluded that mifepristone was no more effective than placebo, regardless of the dose (50 to 600 mg) (N. Berkane et al, Use of mifepristone to ripen the cervix and induce labor in term pregnancies, American Journal of Obstetrics and Gynecology (2005) 192, 114-20).

There is thus a need to adapt to the different treatments while having a good efficiency at ripening the cervix and induce labour.

### SUMMARY OF THE INVENTION

The present invention provides new dosages of a mifepristone oral formulation for use in the induction of labour.

Indeed, as demonstrated above, there is insufficient information available from clinical trials to support the use of mifepristone to induce labour and very little information available on the effects on the baby. In addition, the existing dosage cannot be adjusted to all women.

More specifically, the object of the invention is a mifepristone oral formulation comprising an amount of mifepristone selected from the group consisting of 75 mg, 150 mg or 300 mg, for use in the induction of labour in a pregnant woman with at least 37 weeks gestation and an unfavourable cervix with a Bishop score of between 0 to 6.

Advantageously, the pregnant woman can be at least 40 weeks gestation, preferably at least 41 weeks gestation.

Advantageously, said pregnant woman had a previous Caesarean section, preferably said mifepristone oral formulation comprises 75 mg of mifepristone.

In a first embodiment, the mifepristone oral formulation for its use according the invention comprises 75 mg or 150 mg of mifepristone when said pregnant woman is multiparous.

Preferably, said pregnant woman is multiparous when she has experienced 2 or more previous childbirths.

Preferably, the mifepristone oral formulation for its use according to the invention comprises 75 mg of mifepristone when said pregnant woman is multiparous and already had a Caesarean section.

In a second embodiment, the mifepristone oral formulation for its use according the invention comprises 150 mg or 300 mg of mifepristone when said pregnant woman is nulliparous. Advantageously, the Bishop score of the pregnant woman can be 5 or less than 5, more preferably 4 or less than 4.

In a third embodiment, the mifepristone oral formulation for its use according the invention comprises 150 mg or 300 mg of mifepristone when said pregnant woman is primiparous.

Preferably, said primiparous pregnant woman has experienced one previous childbirth.

Advantageously, the mifepristone oral formulation for its use according to the invention can be used for its single administration to the pregnant woman.

Advantageously, the mifepristone oral formulation for its use according to the invention can be further administered to the pregnant woman 24 hours±6 hours or 48 hours±6 hours after the first administration if the Bishop score is still less than or 6.

Advantageously, the mifepristone oral formulation for its use according to the invention can increase the Bishop score at least 24 hours±6 hours after the first administration.

In a preferred embodiment, the mifepristone oral formulation for its use according to the invention can increase the Bishop score of the pregnant woman of 2 or at least 2.

Advantageously, the induction of labour is outpatient.

Advantageously, the mifepristone oral formulation for its use according to the invention can be used in combination with at least one further therapeutic agent or manual treatment chosen amongst prostaglandins, prostaglandins analogues, oxytocin, balloon catheter, manual dilatation, and their combination.

Advantageously, the mifepristone oral formulation for its use according to the invention can be a solid oral form chosen amongst a tablet, a capsule, an oral dispersible dosage form, a sachet, granules, a powder.

In a preferred embodiment, the mifepristone oral form is a tablet, preferably a breakable tablet.

Advantageously, the mifepristone oral formulation for its use according the invention can comprise further at least one pharmaceutical acceptable excipient chosen amongst carriers, fillers, diluents, preservative, biding agents, wetting agent, dispersible agents, sweetener, flavouring agents, colouring agents, flow agents.

In the most preferred embodiment, the mifepristone oral formulation of the invention is a tablet, preferably a breakable tablet, and further comprises corn starch, povidone (E1201), magnesium stearate, anhydrous colloidal silica and microcrystalline cellulose.

### DETAILED DESCRIPTION OF THE INVENTION

The present invention provides new dosages of a mifepristone oral formulation.

The first object of the invention is a mifepristone oral formulation comprising an amount of mifepristone selected from the group consisting of 75 mg, 150 mg or 300 mg, for use in the induction of labour in a pregnant woman with at least 37 weeks' gestation and an unfavourable cervix with a Bishop score of between 0 to 6.

Mifepristone (CAS number: 84371-65-3), also known as RU-486, is a steroidal antiprogestogen of the following formula:

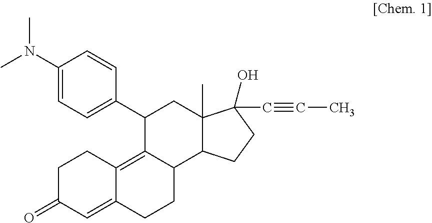 [SMILES: CC#CC1(O)CCC2C3CCC4=CC(=O)CCC4=C3C(c3ccc(N(C)C)cc3)CC21C]

In the presence of progesterone, it acts as a competitive progesterone receptor antagonist. As a result of the withdrawal of the inhibitory effect of progesterone, there is an increase in the synthesis of prostaglandins. Sensitivity of the myometrium to the contraction inducing activity of prostaglandins markedly increased after mifepristone administration.

According to the present invention, the dosages of mifepristone are useful for inducing labour in a pregnant woman who is at term, i.e. a woman who is at least 37 weeks pregnant, preferably at least 40 weeks, more preferably 41 weeks. The dosages of mifepristone oral formulation according to the invention are specifically useful for ripening the cervix and thus inducing labour in full-term women when medically indicated.

Preferably, the Bishop score of the pregnant woman can be 6 or less than 6 more preferably 5 or less than 5 and even more preferably 4 or less than 4. The Bishop score, also known as Pelvic Score, is commonly used to rate the readiness of the cervix for induction of labour. The Bishop Score gives points to 5 measurements of the pelvic examination: dilation, effacement of the cervix, station of the foetus, consistency of the cervix, and position of the cervix. Each measurement is given a sub-score between 0 and 3 as follows:

TABLE 1

| Cervical Exam | 0 point | 1 point | 2 points | 3 points |
|---|---|---|---|---|
| Dilation | Closed | 1-2 cm | 3-4 cm | 5-6 cm |
| Effacement (%) | 0-30% | 40-50% | 60-70% | 80% and |
| | | | | more |
| Station | −3 | −2 | −1, 0 | +1, +2 |
| Consistency of the | Firm | medium | soft | |
| cervix | | | | |
| Position of cervix | posterior | Mid | anterior | |

According to an embodiment of the invention, said pregnant woman is multiparous and had a previous Caesarean section, preferably said mifepristone oral formulation comprises 75 mg of mifepristone.

In a first embodiment, the mifepristone In a first embodiment, the mifepristone oral formulation for its use according the invention can comprise 75 mg or 150 mg of mifepristone when said pregnant woman is multiparous. Multiparous is the medical term for a woman having experienced one or more previous childbirths. However, within the context of the present invention, multiparous designates a woman having experienced two or more previous childbirths. According to the first embodiment, the mifepristone oral formulation for its use according the invention can comprise 75 mg or 150 mg of mifepristone when said pregnant woman has experienced 2 or more previous childbirths.

In a second embodiment, the mifepristone oral formulation for its use according the invention can comprise 150 mg or 300 mg of mifepristone when said pregnant woman is nulliparous. Nulliparous is the medical term for a female that has not borne offspring.

In a third embodiment, the mifepristone oral formulation for its use according to the invention can comprise 150 mg or 300 mg of mifepristone when said pregnant woman is a primiparous. Primiparous is the medical term for a female that has borne only one offspring. According to the third embodiment, the mifepristone oral formulation for its use according the invention can comprise 150 mg or 300 mg of mifepristone when said pregnant woman has experienced one previous childbirth.

The mifepristone oral formulation for its use according to the invention can be used for its single administration to the pregnant woman. The mifepristone oral formulation of the invention can be dosed only once to the pregnant woman the day of her arrival to labour induction. In this embodiment, the single administration is enough to induce cervix ripening and no further administration of said mifepristone oral formulation, therapeutic agent or manual treatment is required for labour induction.

The mifepristone oral formulation for its use according to the invention can be further administered to the pregnant woman 24 hours±6 hours or 48 hours±6 hours after the first administration if her Bishop score is still less than or 6.

Advantageously, the mifepristone oral formulation for its use according to the invention can increase the Bishop score at least 24 hours±6 hours after the first administration.

According to an aspect of the invention, the mifepristone oral formulation comprising an amount selected from the group consisting of 75 mg, 150 mg or 300 mg mifepristone, and said formulation is for use in the induction of labour in a pregnant woman with at least 37 weeks' gestation and an unfavourable cervix with a Bishop score of 6 or less than 6, and wherein:
at Day 0: mifepristone is administered once to said pregnant woman, and if no signs of delivery
optionally at Day 1: the Bishop score of said pregnant woman is determined, and/or
optionally at Day 2: the Bishop score of said pregnant woman is determined,
   and when a Bishop score of above 6 is observed, the pregnant woman will be treated with labour inducing compounds or methods according to a standard protocol. Standard protocols can be found in the WHO recommendations for induction of labour (ISBN: 978 92 4 150115 6) or adopted by the physician.

According to an embodiment, the mifepristone oral formulation comprising an amount selected from the group consisting of 75 mg, 150 mg or 300 mg mifepristone is for use in the induction of labour in a pregnant woman with at least 37 weeks gestation and an unfavourable cervix with a Bishop score of 6 or less than 6, and wherein said mifepristone oral formulation is administered only once to said pregnant woman on Day 0. Day 0 is the day of her arrival for the induction of the labour. In this embodiment, the mifepristone oral formulation is sufficient to induce and induce the labour in said pregnant woman within 1 or 2 days, without the further administration of therapeutic agents or manual treatment conventionally used for labour induction.

According to another embodiment, the mifepristone oral formulation comprising an amount selected from the group consisting of 75 mg, 150 mg or 300 mg mifepristone is for use in the induction of labour in a pregnant woman with at least 37 weeks gestation and an unfavourable cervix with a Bishop score of 6 or less than 6, and wherein said mifepristone oral formulation is administered a first time at Day 0, and if no sign of delivery, the Bishop score of said woman is determined at Day 1 and/or Day 2, and said mifepristone oral formulation can be further administered at Day 1 or Day 2. In this embodiment, the pregnant woman receives a first dose of the mifepristone oral formulation of the invention on Day 0. Then the Bishop score can be assessed on Day 1 and/or Day 2. According to the Bishop score of said pregnant woman, the mifepristone oral formulation according to the invention can be further administered either at Day 1, after the assessment of the Bishop score at Day 1 or before the assessment of the Bishop score at Day 2, or be further administered at Day 2, after the assessment of the Bishop score at Day 1 and/or Day 2. The skilled person is able to determine the moment to administer a second dose of the mifepristone oral formulation to said pregnant woman according to the Bishop score.

The mifepristone oral formulation according to the invention allows the ripening of the cervix and improves the Bishop score. Preferably, the mifepristone oral formulation for its use according to the invention can increase the Bishop score of the pregnant woman of 2 or at least 2.

In an embodiment of the invention, the induction of labour is outpatient. Indeed, the pregnant woman can receive the first dose, and possibly the second dose, and wait for the labour to start in the comfort of her home.

Advantageously, the mifepristone oral formulation for its use according to the invention can be used in combination with at least one further therapeutic agent or manual treatment chosen amongst prostaglandins, prostaglandins analogues, oxytocin, balloon catheter, manual dilatation, and their combination. The further therapeutic agent can be used at the usual dosage or carried out conventionally.

The mifepristone oral formulation for its use according to the invention can be a solid oral form chosen amongst a tablet, a capsule, an oral dispersible dosage form, a sachet, granules, a powder. In a preferred embodiment, the mifepristone oral form is a tablet, more preferably a breakable tablet.

The mifepristone oral formulation can further comprise at least one pharmaceutical acceptable excipient chosen amongst carriers, fillers, diluents, preservative, biding agents, wetting agent, dispersible agents, sweetener, flavouring agents, colouring agents, flow agents. The skilled person can use the appropriate pharmaceutical excipients to formulate the oral formulation according to the invention.

In the context of the invention, a “pharmaceutically acceptable” salt or excipient means any salt or excipient authorized by the European Pharmacopoeia (noted “Ph. Eur.”) and the United States Pharmacopoeia (noted “United States Pharmacopeia (USP)”).

In the most preferred embodiment, the mifepristone oral formulation of the invention is a tablet, preferably a breakable tablet, and further comprises corn starch, povidone, magnesium stearate, anhydrous colloidal silica and microcrystalline cellulose.

### Example

A clinical trial is conducted on 150 pregnant women between 18 and 40 years old. The inclusion criteria were:
full-term pregnancy women at week 40+5 days of gestation,
Bishop score ≤5 at day 0 (baseline),
intact membranes,
primiparous women,
BMI before pregnancy ≤30,
singleton physiological pregnancy, and
pregnancy without medical medication (no risk factors, no abnormalities in clinical and laboratory examinations during pregnancy).

The women either receive a 75 mg tablet, a 150 mg tablet or a 300 mg tablet of mifepristone at Day=0 of the trial or a placebo in the control group. The Bishop score is assessed 24 hours after the first administration of either mifepristone or placebo.

The primary endpoints of the study are a gain in Bishop score 48 hours from baseline and the presence or any adverse events. The secondary endpoints are the following:
rate of subjects with Bishop score gain ≤2,
change in Bishop Score after 24 hours,
time to vaginal delivery,
rate of spontaneous vaginal delivery (without any assistance other than mifepristone or placebo in the control group), and rate of Caesarean sections

Regarding the babies, a follow-up study is performed at the day of delivery, day of delivery +1 and day of delivery +2. The follow-up study comprises the assessment of the following measurements:
Apgar Score (1, 5 and 10 minutes after the delivery)
Vital signs
Physical examination
Newborn screening tests (day of delivery +1)
Dosage of mifepristone and metabolites in umbilical cord blood (day of delivery)
Dosage of ACTH, cortisol and aldosterone in umbilical cord blood (day of delivery)
Concomitant medication,
Adverse event recording.

The Apgar score is a method to quickly assess the health of new-born child. The Apgar score is determined by evaluating the new-born baby on five simple criteria on a scale from zero to two, then summing up the five values, as shown in Table 2. The resulting score thus ranges from zero to 10. The five criteria are summarized using words chosen to form a backronym (Appearance, Pulse, Grimace, Activity, Respiration).

TABLE 2

| Parameter | Score of 0 | Score of 1 | Score of 2 |
|---|---|---|---|
| Skin color | blue or pale | blue at extremities, | no cyanosis body |
| (Appearance) | all over | body pink | and extremities |
| | | (acrocyanosis) | pink |
| Pulse rate | Absent | <100 beats per | ≥100 beats per |
| (Pulse) | | minute | minute |
| Reflex | No response to | grimace on suction | cry on stimulation |
| irritability | stimulation | or aggressive | |
| (Grimace) | | stimulation | |
| Muscle tone | None | Some flexion | flexed arms and |
| (Activity) | | | legs that resist |
| | | | extension |
| Respiratory | absent | weak, irregular, | strong, robust cry |
| effort | | gasping | |
| (respiration) | | | |

Another evaluation of the baby's health is carried out at the age of 5 months. Local paediatricians are asked to complete a questionnaire containing the information on psychomotor development, medication, any pathology in laboratory findings or any examinations, and adverse events.

Statistical analysis: a statistical analysis is done on the treatment difference for the gain in Bishop score at 48 hours from baseline The 2-sided 95% confidence interval is calculated and reported to detect a statistically significant and clinically relevant treatment effect.

1-10. (canceled)

11. A method of inducing labor in a pregnant woman with at least 37 weeks gestation and an unfavourable cervix with a Bishop score of between 0 to 6, the method comprising administering to the pregnant woman a mifepristone oral formulation comprising an amount of mifepristone selected from the group consisting of 75 mg, 150 mg, and 300 mg.

12. The method according to claim 11, wherein the pregnant woman is with at least 40 weeks gestation.

13. The method of claim 11, wherein the mifepristone oral formulation comprises 75 mg or 150 mg of mifepristone and the pregnant woman is multiparous.

14. The method of claim 11, wherein the pregnant woman has had a previous Caesarean section.

15. The method of claim 14, wherein the mifepristone oral formulation comprises 75 mg of mifepristone.

16. The method of claim 11, wherein the pregnant woman is multiparous and the mifepristone oral formulation comprises 150 mg or 300 mg of mifepristone.

17. The method of claim 11, wherein the pregnant woman is primiparous and the mifepristone oral formulation comprises 150 mg or 300 mg of mifepristone.

18. The method of claim 11, wherein the mifepristone oral formulation is administered in a single administration to the pregnant woman.

19. The method of claim 11, wherein the mifepristone oral formulation is further administered to the pregnant woman 24 hours±6 hours or 48 hours±6 hours after a first administration if her Bishop score is less than 6.

20. The method of claim 11, wherein the mifepristone oral formulation is administered in combination with a manual treatment selected from the group consisting of balloon catheter, manual dilatation, and combinations thereof, or with at least one further therapeutic agent selected from the group consisting of prostaglandins, prostaglandin analogues, and oxytocin.

21. The method of claim 11, wherein the induction of labour is carried out on an outpatient.
